(19) United States
(12) Patent Application Publication
(10) Pub. No.: US 2017/0333120 A1
(43) Pub. Date: Nov. 23, 2017

(54) SURGICAL DEVICE HAVING CONSTRAINED ELECTRODE AND METHOD OF USE

(71) Applicant: Corinth MedTech, Inc., Cupertino, CA (US)

(72) Inventor: Csaba Truckai, Saratoga, CA (US)

(73) Assignee: Corinth MedTech, Inc., Cupertino, CA (US)

(21) Appl. No.: 15/603,211

(22) Filed: May 23, 2017

Related U.S. Application Data
(60) Provisional application No. 62/340,445, filed on May 23, 2016.

(51) Int. Cl. A61B 18/14; A61B 1/00; A61B 5/03; A61B 18/00

(52) U.S. Cl. CPC A61B 18/1485; A61B 5/036; A61B 1/00128; A61B 2018/1475; A61B 2018/1405; A61B 2018/00547; A61B 2018/00208; A61B 2018/00083; A61B 2218/002; A61B 2218/007; A61B 2018/00601

(57) ABSTRACT

A tissue resecting device includes an elongated shaft having a central axis, a distal end, and a proximal end. A ceramic or other housing is mounted at the distal end of the shaft and has a tissue-receiving window. A movable electrode is configured to be rotationally oscillated or otherwise moved across the window. In one instance, the rotatable moveable electrode may have a dogleg configuration with a free end constrained within an arcuate slot formed near the window. In another instance, the movable electrode may have a U-shaped configuration with a distal end coupled to a pivot in the housing which is aligned with a rotational drive member.

### CROSS-REFERENCE TO RELATED APPLICATIONS

This application claims the benefit of provisional application No. 62/340,445 (Attorney Docket No. 42005-707.101), filed on May 23, 2016, the full disclosure of which is incorporated herein by reference.

### BACKGROUND OF THE INVENTION

1. Field of the Invention. The present invention relates to devices and methods for resecting and removing tissue from the interior of a patient's body, for example in a transurethral resection of prostate tissue to treat benign prostatic hyperplasia.

Electrosurgical cutting devices often comprise a shaft or sleeve having a tissue extraction lumen with one or more radio frequency (RF) cutting blades arranged to resect tissue which may then be drawn into the extraction lumen, often via vacuum assistance through a cutting window. Most such electrosurgical tissue cutting devices rely on manually engaging the cutting window against the target tissue to be resected. While such manual engagement is often sufficient, in other cases, such as in laparoscopic procedures having limited access and field of view, the target tissue can be difficult to visualize prior to resection and, in particular, it can be difficult to assure that the optimum target site has been engaged by the cutting window. For these reasons, it would be desirable to provide improved electrosurgical cutting tools having improved visibility and ability to engage and immobilize tissue prior to cutting and to extract the tissue from tools after cutting.

US2017/0105748, commonly owned with the present application and incorporated herein by reference herein, describes an improved electrosurgical cutting device comprising an elongated shaft having a central axis, a distal end, and an outer surface. An offset housing is mounted on the distal of the shaft and has a tissue-receiving window. The tissue-receiving window is offset radially outwardly from the outer surface of the shaft, and a moveable electrode is configured to oscillate back and forth across the window to resect tissue which extends into the window. The offset housing improves visibility of the cutting window when viewed from endoscopes and other visualization apparatus.

While a substantial improvement over earlier electrosurgical cutting devices, the moveable electrode of the device of US2017/0105748 has a free distal end that is free-floating and which in rare instances can be caught by tissue and be lifted away from the ceramic housing. In other rare instances, the wire-like shaft of the devices of US2017/0105748 can potentially twist to an unwanted degree when the electrode engaged dense tissue.

For these reasons, it would be desirable to provide improved electrosurgical cutting devices, of the type generally taught in US2017/0105748, where the motion of the moveable electrode is more stabilized and the shaft is less prone to twisting. At least some of these objectives will be met by the inventions described below.

2. Description of the Background Art. US2017/0105748 has been discussed above. Other related patents and published applications include U.S. Pat. No. 8,221,404; U.S. Pat. No. 7,744,595; U.S. Pat. Publ. 2014/0336643; U.S. Pat. Publ. 2010/0305565; U.S. Pat. Publ. 2007/0213704; U.S. Pat. Publ. 2009/0270849; and U.S. Pat. Publ. 2013/0090642.

### SUMMARY OF THE INVENTION

The present invention provides improved devices and methods for resecting tissue. Devices according to the present invention include an elongated member having a ceramic or other housing at its distal end. The elongated member typically has an axial lumen extending between distal and proximal ends, and the lumen typically receives a rotatable shaft having a distal end which terminates near the window in the housing and a proximal end which is configured to connect to a motor, typically located in a handle at a proximal end of the elongated member. A moveable electrode is coupled to a distal end of the rotatable shaft, and the electrode usually comprises an active portion which is radially offset from a central axis of the rotatable shaft so that rotational oscillation of the rotatable shaft causes the active portion to reciprocate, i.e. sweep back-and-forth, across the window in the housing.

In a first aspect of the present invention, the rotatable shaft comprises a tubular member having sufficient torsional strength or “stiffness” to resist twisting of the shaft while it is being rotationally oscillated or otherwise driven by the motor. In particular, the tubular member may comprise a rigid typically metal tube, usually having an insulative (electrically insulating) outer surface, often comprising a stainless steel tube covered by the insulated outer coating, sleeve, or the like. In more specific examples, the insultive outer surface may comprise a heat shrink polymer.

It has been found that a tubular member, typically a stainless steeled tubular member, having a wall thickness of at least about 0.005 in., more typically at least about 0.010 in., will be sufficient to provide torsional strength necessary to resist twisting of the shaft during motor driven movement.

In other specific aspects of this first example of the tissue resecting device of the present invention, the moveable electrode will have an active portion that extends across the tissue-receiving window with a profile that is substantially smaller than the window area. In this way, the active portion of the electrode will still leave a sufficient cross-sectional area of the window open to permit fluid aspiration around and past the active portion of the electrode even while the electrode is moving relative to the window.

In still further specific examples of this first example of the tissue resecting device, the motor may be configured to drive the active portion of the electrode at an oscillatory rate equal to or greater than 1 cycle per second (CPS) relative to the window. In many instances, the oscillatory rate will be equal to or great than 5 CPS relative to the window.

In still further specific aspects of this tissue resecting device, the active portion of the electrode may be offset outwardly from an axis of the rotatable electrode shaft by a distance of at least 2 mm, often by a length of at least 4 mm.

In a second aspect or example of the present invention, a tissue resecting device comprises an elongated member having a proximal end and a distal end. A housing is located at the distal end of the elongated member, and the housing has a tissue-receiving window through a side portion or wall thereof. Both the elongated member and the housing are typically hollow and have lumens therethrough where the lumen in the elongated member is aligned with the lumen in the housing. In this way, a continuous path is formed from the window in the housing to the proximal end of the elongated member. A rotatable shaft extends axially through the elongated member, typically through the lumen thereof, from the proximal end to the distal end of the housing. A movable electrode is coupled to a distal end of the rotatable shaft. A means for constraining the movable electrode is provided so that the electrode will move across the window in a fixed path as the rotatable shaft is rotated.

In a first specific embodiment, the means for constraining the movable electrode comprises a constraining channel located adjacent to a distal end of the window in the housing. A distal tip of the movable electrode travels in the constraining channel as the rotatable shaft is rotationally oscillated about its axis. Typically, at least an active of the movable electrode is radially offset from a rotational axis of the rotatable shaft. The constraining channel will usually have an arcuate path with a radius equal to the distance of the radial offset, and in this way the active portion of the electrode is able to track in the constraining channel as it is rotationally oscillated.

In such specific embodiments where the distal tip follows in a constraining channel, the movable electrode is usually a continuous element with a “dogleg” shape with one end attached to the elongated member and a free end (the distal tip) traveling in the constraining channel. By “dogleg” shape, it is meant that the movable electrode will have a first straight portion disposed along an axis and an active portion radially offset from the first straight portion. The active portion will usually be aligned with the first straight portion along a parallel axis. The active portion will usually also be straight, but in other instances could be slightly curve or have irregular profiles. A lateral portion or segment of the movable electrode joins the first straight portion and the active portion so that the movable electrode is an integrated structure capable of conducing electricity, typically being an electrically conductive metal. At least the active portion of the movable electrode will be exposed to engage or contact tissue and deliver electrosurgical current, usually a cutting current, while other portions not intended to contact tissue may be covered with an insulating sleeve or other material.

In a second specific embodiment, the constraining means may comprise a fixed pivot in a distal end of the housing where the pivot is axially aligned with an axis of the rotatable shaft. A distal tip of the movable electrode is axially aligned with the axis of the rotatable shaft and is rotatably coupled to the fixed pivot, and the active portion of the movable electrode is radially offset from the axis of the rotatable shaft, usually being formed with U-shaped deflection in a continuous metal element. A forward or free end of such the rotatable shaft can be rotatably coupled to the fixed pivot in order to constrain movement of the active portion of the movable electrode as the rotatable shaft is rotated. In particular, the active portion of the movable electrode will circumscribe an arcuate path with a center of rotation defined by the axis of the rotatable member. The arcuate path will usually span the entire width of the window and in some instances will extend beyond the sides or lateral edges of the window. The span of arcuate travel will usually be at least 5°, more usually being at least 10°, and frequently being in the range from 5° to 60°, often from 10° to 40°.

Such tissue resecting devices will usually further comprise a motor configured to rotationally oscillate the rotatable shaft in order to move the electrode. The motor will typically be located in a handle permanently or removable attached to a proximal end of the elongated member. For example, the movable electrode may be adapted to move or reciprocate from side-to-side across the window. The motor drive may be further configured to oscillate the active portion of the electrode at a rate greater than or equal to 1 CPS and the window on the housing may be radially offset outwardly from the outer surface of the elongate member by at least 2 mm, frequently by at least 4 mm.

The window in the ceramic or the housing may have two laterally spaced-apart edges or sides, where the movable electrode may further have a range of movement that extends the active portion thereof past these sides of the windows. Such sides of the windows may alternatively or additionally include ledges for receiving the electrode.

All embodiments of the present invention will usually be configured to be attached to a negative pressure or vacuum source to in order to extract resected tissue through tissue extraction lumens in the elongated member and the housing. Usually but not necessarily, the rotatable shaft will also be located at least partially in such tissue extraction lumens, but in other instances the rotatable shaft may be located in separate lumen(s) in the elongated member and the housing.

### BRIEF DESCRIPTION OF DRAWINGS

FIG. 1 is a view of a tissue resecting device and a block diagram of systems and operating components corresponding to the invention.

FIG. 2 is a perspective view of the working end of the resecting device of FIG. 1 showing an asymmetric ceramic housing and moving electrode that is adapted to sweep across a tissue-receiving window.

FIG. 3 is another perspective view of the working end of the resecting device of FIG. 1 from a different angle.

FIG. 4A is a schematic view of the working end of FIGS. 2-3 interfacing with tissue targeted for resection under endoscopic vision.

FIG. 4B is a schematic view of a working end of a prior art tubular cutting device used in a hypothetical resection procedure.

FIG. 5 is another schematic view of the working end of FIGS. 2-3 being used to resect targeted tissue to a significant depth from the organ surface.

FIG. 6 is a perspective view of a distal dielectric housing of a working end similar to that of FIGS. 2-3 showing window sides with ledges for receiving the electrode at the ends of its movement in a sweeping arc.

FIG. 7A is a perspective view of a distal ceramic housing of a working end similar to that of FIG. 6 with the distal tip of the moveable electrode adapted to move in a constraining slot or channel.

FIG. 7B is a perspective view of an alternative ceramic housing similar to that of FIG. 7A with the distal tip of the moveable electrode adapted to pivot or rotate in a bore or pivot.

### DETAILED DESCRIPTION OF THE INVENTION

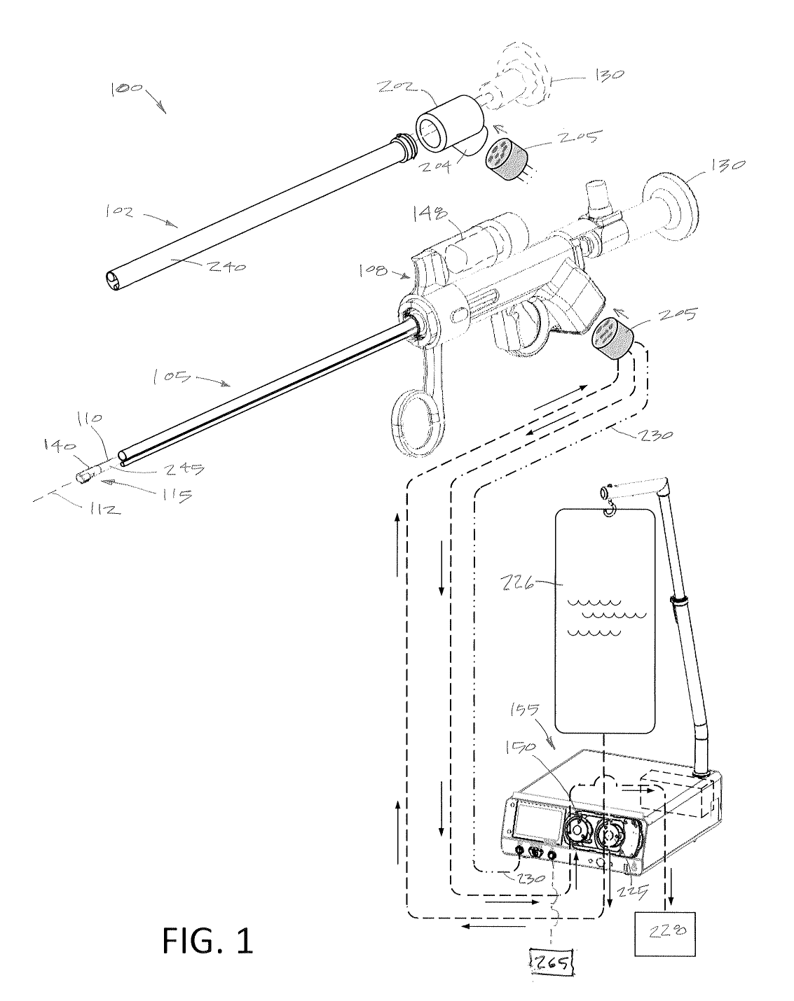

FIGS. 1 illustrates an electrosurgical tissue resecting system 100 for use in urological procedures to resect tissue that includes an introducer sleeve or sheath 102 and a hand-held single-use tissue resecting device or probe 105. The resecting device 105 has a handle portion 108 that is coupled to an elongated shaft or extension portion 110 that has an outer diameter ranging from about 2 mm to 7 mm, and in one variation is 5 mm in diameter. The shaft 110 extends about longitudinal axis 112 to a working end 115 that is radially asymmetric relative the shaft 110 and its axis 112 as further described below. In one variation, the device is adapted for performing a TURP procedure (transurethral resection of prostate) or a bladder tumor resection procedure and thus the shaft portion 110 extends about axis 112 with a length suitable for introducing in a transurethral approach to reach the targeted prostate tissue or bladder tissue.

As will be described below and shown in FIG. 1, the resecting device 105 is adapted for introduction through the introducer sleeve 102. Such an introducer sleeve 102 is adapted to receive a commercially available endoscope 130 as can be understood from FIG. 1.

Referring to FIGS. 1-3, in general, it can be seen the resecting device 105 has an elongated shaft 110 that extends to a distal shaft portion 132 that is coupled to an offset resecting housing 140 that has an offset tissue-receiving window 144. A moveable electrode 145 is adapted to be driven by a motor drive unit 148 in handle 108 (see FIG. 1) so that the longitudinal portion 149 of the electrode 145 sweeps across the window 144 from side to side to electrosurgically resect tissue that is captured in the window 144. The targeted tissue can be suctioned into and captured in window 144 by means of a negative pressure source or outflow pump 150 in controller 155 that communicates with a tissue extraction channel 158 extending through the device 105 and terminating in the window 144.

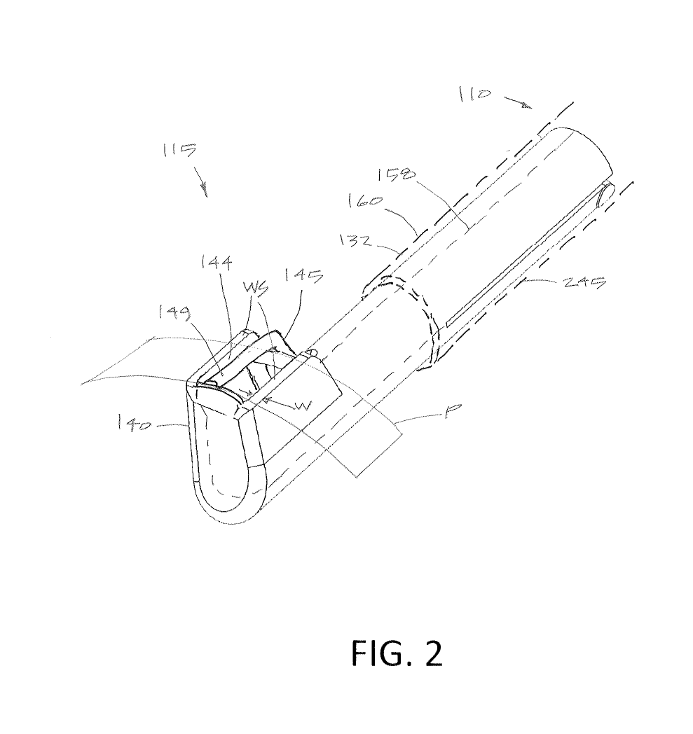

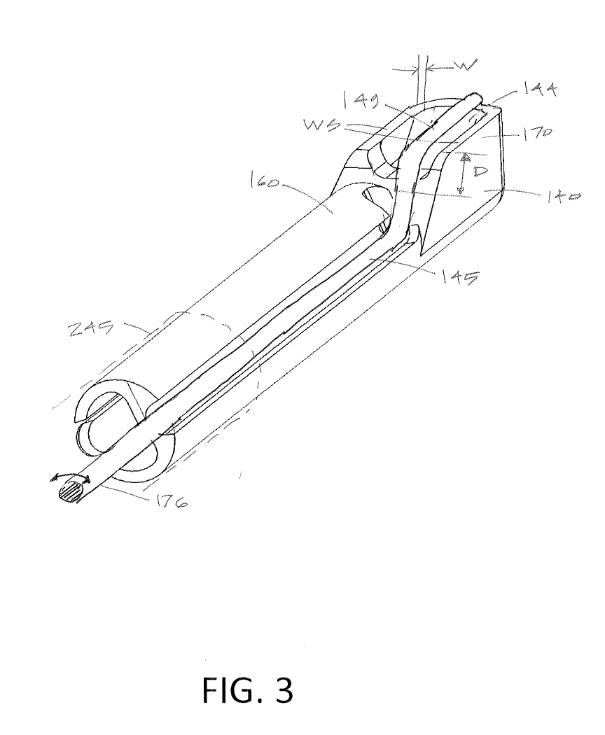

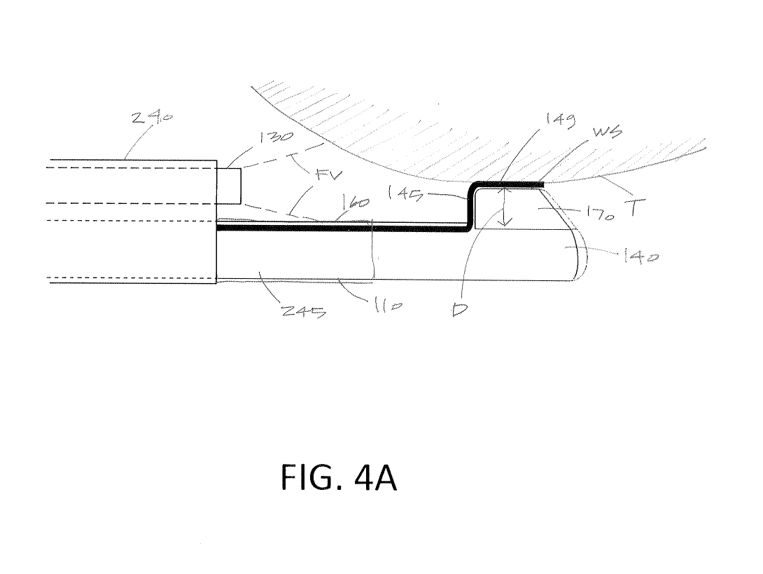

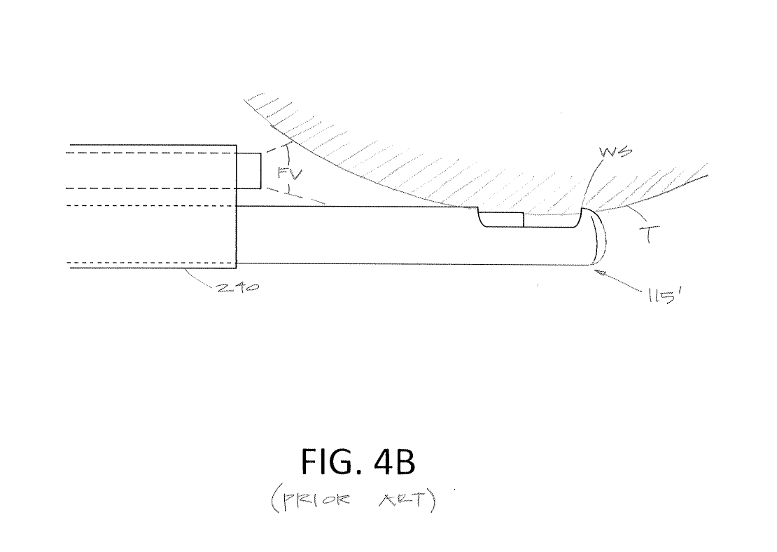

More in particular, referring to FIGS. 2 and 3, the configuration of the offset housing 140 is adapted to perform multiple functions. First, the offset housing 140 positions the window surface WS (within curved plane P indicated in FIG. 2) outwardly from the outer surface 160 of shaft 110 which then allows the window surface WS to be fully visible through a endoscope 130 or other viewing means that would be introduced parallel to the device shaft 110 (see FIG. 4A). For example, FIG. 4A is a schematic view of the working end 115 with working surface WS in contact with targeted tissue T. As can be seen in FIG. 4A, the endoscope 130 is positioned with the field of view FV directly aligned with the working surface WS thus allowing optimal viewing of the tissue resection process.

In contrast, FIG. 4B shows a working end 115′ of a conventional dual sleeve tubular cutter having a window surface WS′ which when pressed against an organ prevents endoscopic vision of the interface between the tubular cutting edge and the tissue T during a resection procedure.

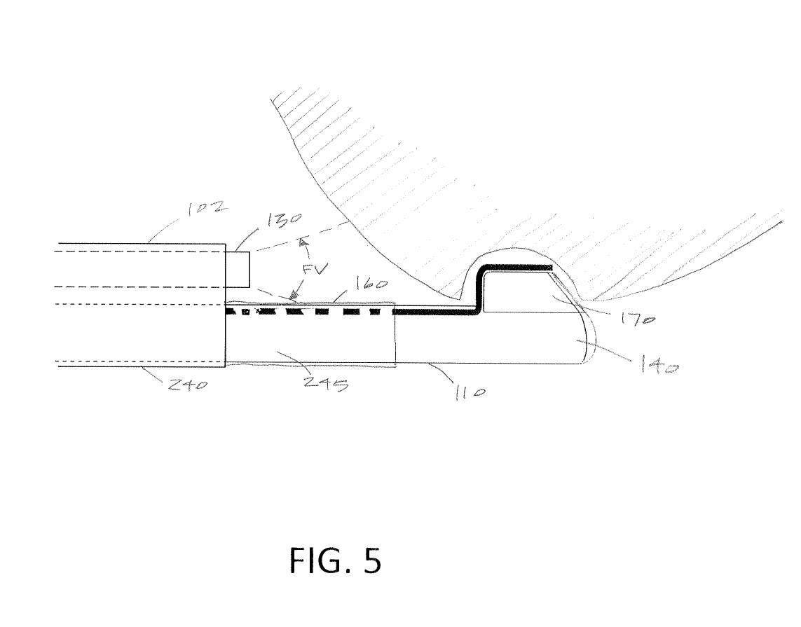

Second, the offset housing 140 is adapted for resecting tissue to a greater depth in a localized region of an organ, rather than resecting surface tissues over a broad area. More in particular as shown in FIG. 5, the offset portion 170 of housing 140 can be pushed into tissue perpendicular to axis 112 of the probe shaft 110. Thus, as shown in FIG. 5, the offset housing 140 can be used to resect tissue deep into in a localized region that would not be possible with a resecting device having the configuration shown in FIG. 4B.

FIGS. 2 and 3 illustrate the asymmetric or offset dielectric housing 140 that can comprise a ceramic material such as zirconium oxide, aluminum oxide or similar materials as is known in the art. In FIGS. 2-3, it can be seen that window surface WS is offset from the shaft surface 160 by a predetermined dimension D which can be from 2 mm to 8 mm and in one embodiment comprises a 5 mm offset.

As can be further be seen in FIGS. 2-3, the width W of the window surface WS around at least portions of the perimeter of the window 144 is a limited dimension, for example less than 3 mm, or less than 2 mm or less than 1 mm. which allows the offset portion 170 of housing 140 to be pushed into tissue perpendicular to the device axis 112 as the electrode 145 sweeps across the window 144.

Referring to FIGS. 2-3, one variation of resecting device 105 has an electrode 145 that can be tungsten or stainless steel wire that with electrode portion 149 adapted to sweep across the window 144 at any suitable rate, for example from 1 CPS (cycles per second) to 50 CPS or more. In FIG. 3, it can be understood that the electrode 145 has an elongated proximal shaft portion 176 that extends into handle 108 of the device (FIG. 1). The proximal end of electrode 145 is operatively coupled to a motor drive unit 148 and a suitable mechanism or controller is provided to rotate the elongated electrode shaft portion 176 in an arc to resect tissue.

As can be understood from FIGS. 2-3, the electrode portion 149 moves back and forth akin to a windshield wiper across window 144 in the offset housing 140. A number of mechanisms can be used to effectuate the desired movements of the electrode, or the motor drive 148 simply can be controlled by software to move in intermittent clockwise and counter-clockwise directions. In one variation, the elongated proximal portion 176 of the electrode 145 will twist over its length and thus the motor drive 148 can be adapted to rotate the electrode shaft in an arc with radial angle which is greater than the window's comparable radial angle or arc. Thus, the electrode portion 149 can be expected to move back and forth entirely across the window even when meeting some tissue resistance by compensating for some twisting that is allowed in the proximal electrode shaft portion 176. In one variation, the motor drive unit can be adapted to over-rotate the electrode shaft portion 176 at its proximal end by a selected amount which can be from 10° radial motion to 90° radial motion to compensate for twisting of the electrode shaft portion to insure that electrode portion 149 sweeps entirely across the surface of window 144.

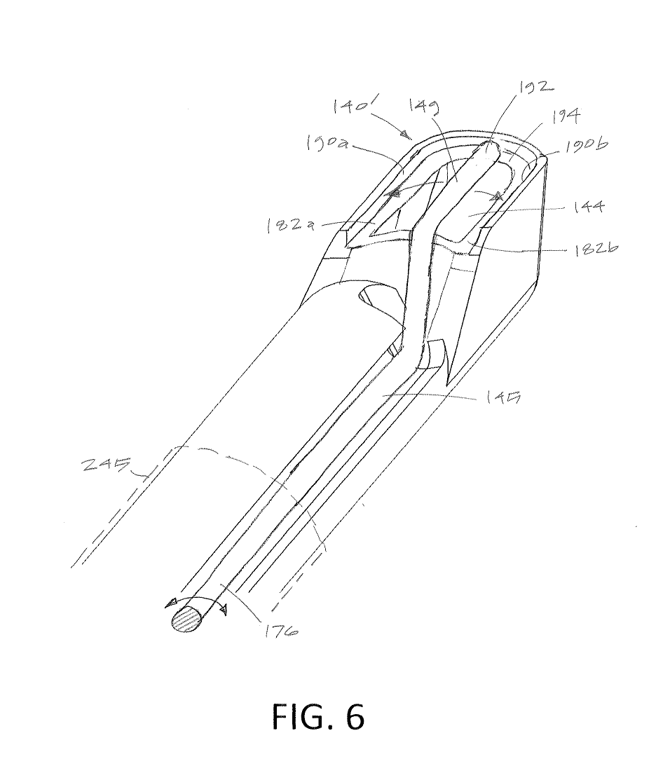

In general, the window 144 in housing 140 can be configured to have a radial arc relative to the electrode shaft 176 ranging between 30° and 180°. In one variation of housing 140′ shown in FIG. 6, it can be seen that the electrode portion 149 has a range of motion that extends across the radial dimension of the window 144 to ensure that any tissue captured in the window is resected as the electrode portion 149 passes the window edges 182a and 182b to function like a shear or in a scissor-like manner. The electrode portion 149 moves over ledges 186a and 186b on either side of the housing 140′ and can bump into surfaces 190a and 190b. By bumping into the surfaces 190a and 190b, any over rotation in the electrode shaft 176 to accommodate twisting as described above can limit the rotation of the electrode portion in the housing 140′. Further, in FIG. 6, it can be seen that the distal tip 192 of electrode portion 149 extends distally beyond window 144 and onto distal ledge 194 in the housing 140′ to ensure tissue is resected by the electrode in the distal window region.

Now turning back to FIG. 1, it can be understood that the resecting device 105 and endoscope 130 can be used with introducer sleeve assembly or sheath 102. As shown in FIG. 1, the introducer assembly 102 has a proximal handle body 202 with a connector 204 that is adapted to couple to connector member 205. The connector 205 is adapted to couple a conduit 206 to controller 155 and provide within a single cable the following: (i) a first lumen communicating with the fluid outflow pump 150, (ii) a second lumen communicating with a fluid inflow pump 225, and (iii) a third lumen communicating with a pressure sensor positioned in the controller 155 or in or near the connector 205. As can be seen in FIG. 1, the introducer sleeve 102 can also accommodate an endoscope 130. Thus, the introducer sleeve 120 can be assembled with the endoscope 130 (and without the resection device 105) and coupled by connector 205 to the controller 155 to provide an inflow of irrigation fluid from fluid source 226, and outflow of irrigation fluid to collection reservoir 228 together with pressure sensing to allow the assembly to be used in a diagnostic procedure prior to a tissue resection procedure. In other words, the introducer sleeve 102 can function as a ‘continuous flow’ optical introducer for use in trans-urethral access to a targeted sire in the prostate or bladder.

After the introducer sleeve assembly 102 is used for an initial diagnostic procedure, the endoscope 130 can be removed from the assembly 102 and connector 205 can be disconnected from handle body 205. Thereafter, the sleeve portion 240 (see FIG. 1) of introducer assembly 102 can be detached from proximal handle body 204 with the sleeve portion 240 remaining in the patient. Next, the endoscope 130 and connector 205 can be assembled with the resecting device 105 and the physician can insert the resecting device 105 through the sleeve portion 240 remaining in the patient to access the targeted site. The resecting device 105 and sleeve portion 204 in combination then provide lumens as described above for fluid inflows, fluid outflows and direct pressure sensing through lumens in connector 205.

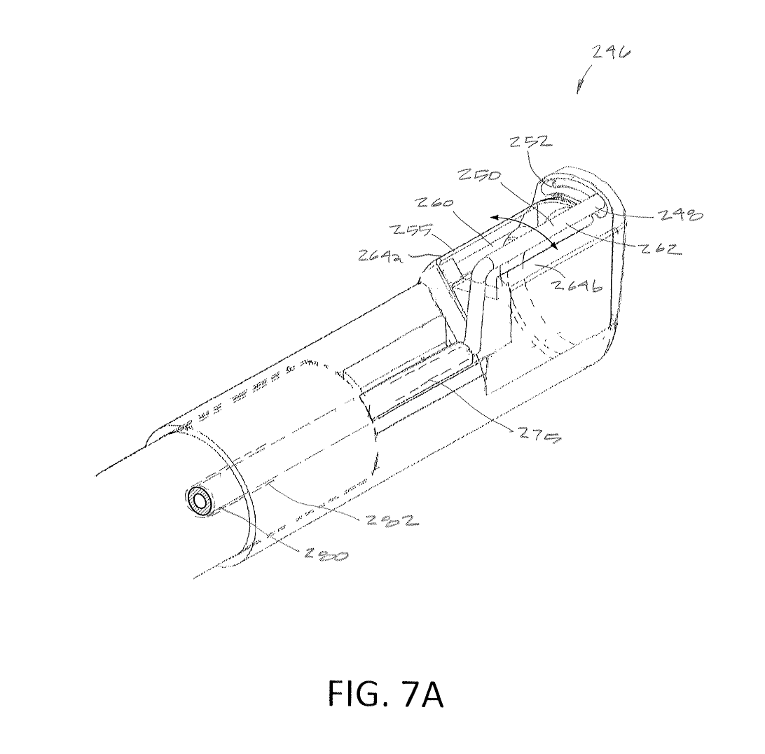

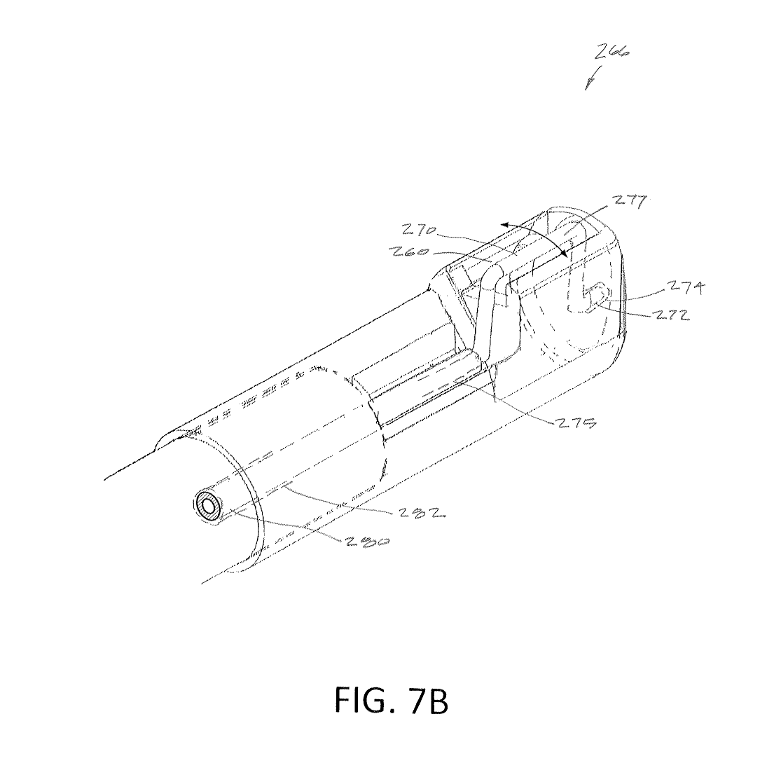

Now turning to FIG. 7A, a perspective view of a distal ceramic housing of a working end 246 similar to that of FIG. 6 is shown. In this variation, the distal tip 248 of the moveable electrode 250 is configured to be constrained within a constraining slot or channel 252. In other words, the distal electrode tip 248 is not free-floating as in the variation of FIG. 6. It has been found that an electrode with a free-floating distal tip can be caught by tissue and be lifted away from the ceramic housing 255. Thus, in this variation the distal electrode tip 248 is constrained and cannot be tangled with tissue or lifted away from the ceramic housing and window 260. The variation of FIG. 7A illustrates an arcuate slot or channel 252 that limits the movement of the electrode 250. In all other respects, the working end functions as described previously. Further, the distal electrode portion 262 and channel 252 can be configured to allow the electrode to pass over the edges 264a and 264b of the window 260 as described above.

FIG. 7B shows another variation of working end 266 in which the electrode 270 has a distal tip 272 that is constrained in a pivot or bore indicated at 274. In this variation, it can be seen that the electrode 270 has a U-shape with the distal tip 272 aligned with the electrode shaft portion 275 to allow the active electrode portion 277 to move from side to side relative to window 260 as described previously.

In another aspect of the invention shown in FIGS. 7A-7B, the electrode shaft portion 275 comprises a tubular member 280 which can comprise a metal hypotube, such as stainless steel or a similar material. In a previous variation as shown in FIG. 6, the electrode shaft portion comprised a wire element which could potentially twist to an unwanted degree when the electrode engaged dense tissue, for example. In this variation, it has been found that a metal hypotube with a suitable wall thickness can resist twisting when the electrode is being moved and engaging dense tissue. In one variation, the wall thickness of the tubular member 280 can be at these 0.005″ or at least 0.010″.

In general, a tissue resecting device corresponding to the invention comprises an elongated member extending along a longitudinal axis to a distal portion having a window communicating with an aspiration source, an electrode having an electrode shaft with a central axis extending within the elongated member to an electrode working end wherein a portion of the electrode working end is offset from said central axis, and a motor configured to rotate the electrode shaft to cause the electrode working end to move relative to the window wherein the electrode shaft comprises a tubular member adapted to resist twisting of said shaft during motor driven movement thereof. Further, the tubular member can comprise a metal tube with an insulative outer surface layer 282. The tissue tubular member can be a stainless steel tube with the insulative outer surface layer comprising a heat shrink polymer.

In one variation, the electrode's working end has a profile that is substantially smaller than the area of the window to thereby permit fluid aspiration around the electrode working end at all times through the window as the electrode is moving relative to the window. This allows the negative pressure source to draw the tissue into the window interface, and maintains the tissue in the interface as the electrode cuts and extracts the resected tissue. In one variation, the electrode working end is motor driven and moves at a rate of equal to or greater than 1 CPS relative to the window, or equal to or greater than 5 CPS relative to the window. As described previously, the electrode working end can be offset radially outward from the shaft assembly by at least 2 mm or by at least 4 mm.

In another aspect of the invention, the tissue resecting device comprises an elongated member extending to a distal housing having a tissue-receiving window, a moveable electrode configured to move across the window, and a motor configured to move the electrode wherein a distal tip of the electrode moves in a constraining channel in the housing. In another variation, the tissue resecting device comprises an elongated member extending to a distal housing having a tissue- receiving window, a moveable electrode configured to move across the window; and a motor configured to move the electrode wherein a distal end of the electrode is non-free floating or pivots in a pivot channel.

What is claimed is:

1. A tissue resecting device comprising:
an elongated member having a proximal end, a distal end, and a longitudinal axis therebetween, said elongated member having lumen therethrough configured to communicate with an aspiration source;
a housing at the distal end of the elongated member, said housing having a tissue-receiving window; and
a rotatable shaft having a central axis and extending axially through the lumen of the elongated member from the proximal end to the housing;
a moveable electrode coupled to a distal end of the rotatable shaft, wherein an active portion of the moveable electrode working is offset from said central axis so that is disposed adjacent to the window; and
a motor configured to rotationally oscillate the rotatable shaft to cause the electrode working end to move relative to the window;
wherein the rotatable shaft comprises a tubular member having sufficient torsional strength to resist twisting of said shaft during motor driven movement.

2. The tissue resecting device of claim 1 wherein the tubular member comprises a metal tube with an insulative outer surface layer.

3. The tissue resecting device of claim 2 wherein the tubular member comprises a stainless steel tube.

4. The tissue resecting device of claim 2 wherein the insulative outer surface layer comprises a heat shrink polymer.

5. The tissue resecting device of claim 1 where the active portion of the electrode has a profile that is substantially smaller than the window area to thereby permit fluid aspiration around said active portion and through the window as the electrode is moving relative to the window.

6. The tissue resecting device of claim 1 wherein the motor is configured to drive the active portion of the electrode at a rate of equal to or greater than 1 CPS relative to the window.

7. The tissue resecting device of claim 6 wherein the rate is equal to or greater than 5 CPS relative to the window.

8. The tissue resecting device of claim 1 wherein the tubular member has a wall thickness of at least 0.005″.

9. The tissue resecting device of claim 1 wherein the tubular member has a wall thickness of at least 0.010″.

10. The tissue resecting device of claim 1 wherein the active portion of the electrode is offset outwardly from the rotatable shaft by at least 2 mm.

11. The tissue resecting device of claim 1 wherein the electrode working end is offset outwardly from the rotatable shaft by at least 4 mm.

12. A tissue resecting device comprising:
an elongated member having a proximal end and a distal end;
a housing at the distal end of the elongated member, said housing having a tissue-receiving window; and
a rotatable shaft extending axially through the elongated member from the proximal end to the housing;
a moveable electrode coupled to a distal end of the rotatable shaft; and
means for constraining the moveable electrode to move across the window in a fixed path as the rotatable shaft is rotated.

13. A tissue resecting device as in claim 12 wherein the means for constraining the moveable electrode comprises a constraining channel located adjacent to a distal end of the window, wherein a distal tip of the moveable electrode travels in the constraining channel as the rotatable shaft is rotationally oscillated.

14. A tissue resecting device as in claim 13 wherein at least an active portion of the moveable electrode is radially offset from a rotational axis of the rotatable shaft and the constraining channel has an arcuate path with a radius equal to the distance of the radial offset.

15. A tissue resecting device as in claim 14 wherein the moveable electrode is a continuous element with a dogleg shape with one end attached to the elongate member and a free end traveling in the constraining channel.

16. A tissue resecting device as in claim 12 wherein the means for constraining the travel path comprises a fixed pivot axially aligned with the rotatable shaft in a distal end of the housing, wherein a distal tip of the moveable electrode is rotatably coupled to the fixed pivot.

17. A tissue resecting device as in claim 16 wherein the moveable electrode is a continuous element with a U-shape with one end attached to the elongate member and a free end rotatably coupled to the fixed pivot.

18. A tissue resecting device as in claim 12 further comprising a motor configured to rotationally oscillate the rotatable shaft to move the electrode.

19. The tissue resecting device of claim 18 wherein the movable electrode is adapted to move from side to side across the window.

20. The tissue resecting device of claim 19 wherein the electrode moves at a rate of equal to or greater than 1 CPS.

21. The tissue resecting device of claim 12 wherein the window is offset outwardly from the outer surface of the elongate member by at least 2 mm.

22. The tissue resecting device of claim 12 wherein the window is offset outwardly from the outer surface of the elongate member by at least 4 mm.

23. The tissue resecting device of claim 12 wherein the window has two laterally spaced-apart sides and the moveable electrode has a range of movement that extends past the sides of the window.

24. The tissue resecting device of claim 19 wherein the laterally spaced-apart sides have ledges for receiving the electrode.

25. The tissue resecting device of claim 12 wherein the housing comprises a ceramic body.
